(19) United States
(12) Patent Application Publication
(10) Pub. No.: US 2019/0083473 A1
(43) Pub. Date: Mar. 21, 2019

(54) AZABICYCLOOCTANE DERIVATIVES AS FXR AGONISTS FOR USE IN THE TREATMENT OF LIVER AND GASTROINTESTINAL DISEASES

(71) Applicant: Novartis AG, Basel (CH)

(72) Inventors: Michael Badman, Newton, MA (US); Lloyd B. Klickstein, Newton, MA (US); Bryan Laffitte, Cardiff, CA (US)

(21) Appl. No.: 16/137,360

(22) Filed: Sep. 20, 2018

Related U.S. Application Data
(63) Continuation of application No. 15/534,324, filed on Jun. 8, 2017.
(60) Provisional application No. 62/093,586, filed on Dec. 18, 2014.

(51) Int. Cl. A61K 31/439; A61K 31/4748; A61K 31/497; A61K 31/426; A61K 31/422; A61K 31/506

(52) U.S. Cl. CPC A61K 31/439; A61K 31/4748; A61K 31/506; A61K 31/426; A61K 31/422; A61K 31/497

(57) ABSTRACT

The invention provides methods for modulating the activity of farnesoid X receptors (FXRs) using compounds of Formula (I) or (II). In particular, the invention provides for the use of compounds of Formula (I) or (II), or a stereoisomer, enantionmer or pharmaceutically acceptable salt thereof, for treating or preventing liver and gastrointestinal diseases.

### FIELD OF THE INVENTION

The present invention relates to methods for treating or preventing a condition mediated by farnesoid X receptors (FXRs).

### BACKGROUND OF THE INVENTION

Farnesoid X Receptor Agonist (FXR) is a nuclear receptor activated by bile acids (Calkin and Tontonoz (2012), Nature Reviews Molecular Cell Biology 13, 213-24). FXR is expressed in principal sites of bile acid metabolism, such as liver, intestine and kidney, where it mediates effects on multiple metabolic pathways in a tissue-specific manner. When activated, FXR affects expression of genes controlling a sensitive, negative feedback loop which controls multiple aspects of bile acid metabolism resulting in reduced bile acid levels (Zollner et al. (2006), Molecular Pharmaceutics 3: 231-51).

In the liver, FXR reduces conversion of cholesterol to bile acids by downregulating the expression of enzymes involved in bile acid synthesis, such as cholesterol 7α-hydroxylase (Cyp7a1) and sterol 12-α hydroxylase (Cyp8b1). FXR also reduces bile acid toxicity in the liver by increasing other bile acid-modifying enzymes including sulphotransferase 2A1 (Sult2a1), UDP-glucuronosyltransferase 2B4 (Ugt2b4) and Cyp3a4. Bile acids are conjugated to either glycine or taurine before secretion into the bile, a process also controlled by FXR. FXR enhances bile acid conjugation by increasing the expression of bile acid CoA synthase (BACS) and bile acid CoA-amino acid N acetyltransferase (BAAT), and FXR promotes the transport of bile acids to the gall bladder via bile salt export pump (BSEP), multidrug resistance protein 2 (MDR2) and MDR3 (Calkin and Tontonoz, supra).

Within the intestine, FXR reduces bile acid absorption via downregulation of the apical sodium-dependent bile acid transporter (ASBT), promotes bile acid movement across the enterocyte via ileal bile acid binding-protein (IBABP) and promotes recycling of bile acids to the liver via organic solute transporter-α (OSTα) and -β (OSTβ). In addition, FXR reduces hepatic uptake of bile acids by reducing the expression of organic anion transporting polypeptide (OATP) and sodium taurocholate cotransporting polypeptide (NTCP). FXR also promotes the release of fibroblast growth factor 15 (FGF15 in rodent; FGF19 in human) from the intestine. FGF15/19 travels to the liver, acting on FGF4 receptor (FGF4R) to reduce Cyp7a1 and Cyp8b1 expression and thus represses bile acid synthesis. Furthermore, FXR affects circulating lipid levels, by reducing lipogenesis via inhibition of sterol-regulatory element-binding protein 1C (SREBP1c) and fatty acid synthase (FAS).

### SUMMARY OF THE INVENTION

The present invention relates to methods for treating or preventing a condition mediated by farnesoid X receptors (FXRs); and more particularly, to the use of FXR agonists or partial agonists for treating or preventing liver disease and gastrointestinal disease.

Various (enumerated) embodiments of the disclosure are described herein. It will be recognized that features specified in each embodiment may be combined with other specified features to provide further embodiments of the present disclosure.

### Embodiment 1

Use of a compound of Formula (I)

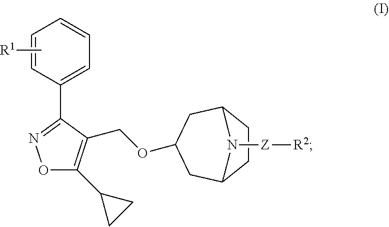

or a stereoisomer, enantiomer, or pharmaceutically acceptable salt thereof;

wherein Z is pyridyl, pyrimidinyl, pyrazinyl, pyridazinyl or benzothiazolyl; each of which is optionally substituted with 1-2 R³ radicals selected from halogen, C₁₋₆ alkyl or C₁₋₆ alkoxy;

R¹ is haloC₁₋₆ alkyl or haloC₁₋₆ alkoxy;

R² is —CO₂R, —CONR—(CR₂)—CO₂R, —CONR—(CR₂)₂—SO₃R or

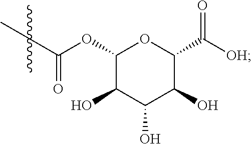 [SMILES: *C(=O)O[C@@H]1O[C@H](C(=O)O)[C@@H](O)[C@H](O)[C@H]1O]

and each R is independently hydrogen or C₁₋₆ alkyl;

or a compound of Formula (II)

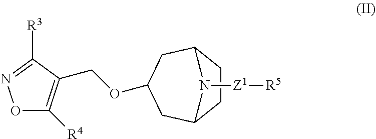

or a stereoisomer, enantiomer, a pharmaceutically acceptable salt, an amino acid conjugate or an acyl glucuronide conjugate thereof;

wherein Z¹ is phenylene, pyridylene, pyrimidinylene, pyrazinylene, pyridazinylene, thiazolylene, benzothiazolyl, benzo[d]isothiazolyl, imidazo[1,2-a]pyridinyl, quinolinyl, 1H-indolyl, pyrrolo[1,2-b]pyridazinyl, benzofuranyl, benzo[b]thiophenyl, 1H-indazolyl, benzo[d]isoxazolyl, quinazolinyl, 1H-pyrrolo[3,2-c]pyridinyl, pyrazolo[1,5-a]pyrimidinyl, imidazo[1,2-b]pyridazinyl, pyrazolo[1,5-a]pyridinyl; each of which is optionally substituted with 1-2 R⁶ radicals selected from halogen, C₁₋₆ alkyl, haloC₁₋₆alkyl, C₁₋₆alkoxy, haloC₁₋₆alkoxy, or cyclopropyl;

R³ is phenyl, pyridyl, bicyclo[3.1.0]hexanyl, spiro[2.3]hexanyl, bicyclo[3.1.1]heptanyl, spiro[2.5]octanyl, bicyclo[4.1.0]heptanyl, bicyclo[3.1.0]hexan-6-yl, spiro[2.3]hexan-5-yl, bicyclo[3.1.1]heptan-3-yl, spiro[2.5]octan-4-yl, bicyclo[4.1.0]heptan-3-yl, cyclohexyl or cyclopentyl, each of which is optionally substituted with 1-3 R^{3a}; or R³ is cyclopropyl optionally substituted with 1-2 R^{3a} or phenyl;

R^{3a} is halogen, C₁₋₆ alkyl, haloC₁₋₆alkyl, C₁₋₆alkoxy, haloC₁₋₆alkoxy or cyclopropyl;

R⁴ is C₁₋₃ alkyl, haloC₁₋₃alkyl or cyclopropyl optionally substituted with C₁₋₃ alkyl or haloC₁₋₃ alkyl;

R⁵ is —X—CO₂R⁷, hydroxyC₁₋₆alkyl, CONR⁷R⁸, CONR(CR₂)₁₋₄CO₂R⁷, CONR(CR₂)₁₋₄SO₃R⁸ or tetrazolyl; wherein X is a bond, C₁₋₂ alkylene or cyclopropyl; and

R, R⁷ and R⁸ are independently hydrogen or C₁₋₆ alkyl;

in the manufacture of a medicament for treating or preventing a condition mediated by Farnesoid X receptor (FXR), wherein said condition is bile acid malabsorption or bile acid diarrhea (e.g. is primary or secondary bile acid diarrhea), bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, or parenteral nutrition-associated liver disease.

### Embodiment 2

A compound of Formula (I) or (II) as defined in Embodiment 1, or a stereoisomer, enantiomer, or a pharmaceutically acceptable salt thereof; and optionally in combination with a second therapeutic agent, for use in treating or preventing a condition mediated by FXR; wherein said condition mediated by FXR is bile acid malabsorption or bile acid diarrhea (e.g. primary or secondary bile acid diarrhea), bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, or parenteral nutrition-associated liver disease.

### Embodiment 3

The use of a compound of Formula (I) according to Embodiment 1, or the compound of Formula (I) for use according to Embodiment 2, wherein R¹ is trifluoromethyl or trifluoromethoxy.

### Embodiment 4

The use of a compound of Formula (I) according to Embodiment 1 or 3, or the compound of Formula (I) for use according to Embodiment 2 or 3, wherein R² in is —CO₂R; and R is hydrogen or C₁₋₆ alkyl.

### Embodiment 5

The use of a compound of Formula (I) according to any one of Embodiments 1 and 3-4, or the compound of Formula (I) for use according to any one of Embodiments 2-4, wherein R³ is methyl, methoxy or fluoro.

### Embodiment 6

The use of a compound of Formula (I) according to any one of Embodiments 1 and 3-5, or the compound of Formula (I) for use according to any one of Embodiments 2-5, wherein Z is pyridyl.

### Embodiment 7

The use of a compound of Formula (I) according to any one of Embodiments 1 and 3-5, or the compound of Formula (I) for use according to any one of Embodiments 2-5, wherein Z is pyrimidinyl.

### Embodiment 8

The use of a compound of Formula (I) according to any one of Embodiments 1 and 3-5, or the compound of Formula (I) for use according to any one of Embodiments 2-5, wherein Z is pyrazinyl.

### Embodiment 9

The use of a compound of Formula (I) according to any one of Embodiments 1 and 3-5, or the compound of Formula (I) for use according to any one of Embodiments 2-5, wherein Z is benzothiazolyl.

### Embodiment 10

The use of a compound of Formula (I) or (II) according to Embodiment 1, or the compound of Formula (I) or (II) for use according to Embodiment 1, wherein said compound of Formula (I) or (II) is selected from:
methyl 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylate;
methyl 2-[3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylate;
2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid;
2-[3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid;
methyl 2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylate;
methyl 2-[3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylate;
2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylic acid;
2-[3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylic acid;
2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-methoxy-1,3-benzothiazole-6-carboxylic acid;
2-[3-({5-cyclopropyl-3-[2-(trifluoromethoxy)phenyl]-1,2-oxazol-4-yl}methoxy)-8-azabicyclo[3.2.1]octan-8-yl]-4-methoxy-1,3-benzothiazole-6-carboxylic acid;
2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-methoxy-1,3-benzothiazole-6-carboxylic acid;
2-[3-({5-cyclopropyl-3-[2-(trifluoromethyl)phenyl]-1,2-oxazol-4-yl}methoxy)-8-azabicyclo[3.2.1]octan-8-yl]-4-methoxy-1,3-benzothiazole-6-carboxylic acid;
ethyl 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-1,3-benzothiazole-6-carboxylate;
ethyl 2-[3-({5-cyclopropyl-3-[2-(trifluoromethoxy)phenyl]-1,2-oxazol-4-yl}methoxy)-8-azabicyclo[3.2.1]octan-8-yl]-1,3-benzothiazole-6-carboxylate;
2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-1,3-benzothiazole-6-carboxylic acid;
2-[3-({5-cyclopropyl-3-[2-(trifluoromethoxy)phenyl]-1,2-oxazol-4-yl}methoxy)-8-azabicyclo[3.2.1]octan-8-yl]-1,3-benzothiazole-6-carboxylic acid;
2-({2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-1,3-benzothiazol-6-yl}formamido)acetic acid;
2-({2-[3-({5-cyclopropyl-3-[2-(trifluoromethoxy)phenyl]-1,2-oxazol-4-yl}methoxy)-8-azabicyclo[3.2.1]octan-8-yl]-1,3-benzothiazol-6-yl}formamido)acetic acid;
2-({2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazol-6-yl}formamido)acetic acid;
2-({2-[3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazol-6-yl}formamido)acetic acid;
2-({6-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyridin-3-yl}formamido)acetic acid;
2-({6-[3-({5-cyclopropyl-3-[2-(trifluoromethoxy)phenyl]-1,2-oxazol-4-yl}methoxy)-8-azabicyclo[3.2.1]octan-8-yl]pyridin-3-yl}formamido)acetic acid;
2-({2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazol-6-yl}formamido)acetic acid;
2-({2-[3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazol-6-yl}formamido)acetic acid;
2-({2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-1,3-benzothiazol-6-yl}formamido)ethane-1-sulfonic acid;
2-({2-[3-({5-cyclopropyl-3-[2-(trifluoromethoxy)phenyl]-1,2-oxazol-4-yl}methoxy)-8-azabicyclo[3.2.1]octan-8-yl]-1,3-benzothiazol-6-yl}formamido)ethane-1-sulfonic acid;
2-({2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazol-6-yl}formamido)ethane-1-sulfonic acid;
2-({2-[3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazol-6-yl}formamido)ethane-1-sulfonic acid;
2-({2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazol-6-yl}formamido)ethane-1-sulfonic acid;
2-({2-[3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazol-6-yl}formamido)ethane-1-sulfonic acid;
2-({6-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyridin-3-yl}formamido)ethane-1-sulfonic acid;
2-({6-[3-({5-cyclopropyl-3-[2-(trifluoromethoxy)phenyl]-1,2-oxazol-4-yl}methoxy)-8-azabicyclo[3.2.1]octan-8-yl]pyridin-3-yl}formamido)ethane-1-sulfonic acid;
methyl 6-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyridine-3-carboxylate;
methyl 6-[3-({5-cyclopropyl-3-[2-(trifluoromethoxy)phenyl]-1,2-oxazol-4-yl}methoxy)-8-azabicyclo[3.2.1]octan-8-yl]pyridine-3-carboxylate;
6-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyridine-3-carboxylic acid;
6-[3-({5-cyclopropyl-3-[2-(trifluoromethoxy)phenyl]-1,2-oxazol-4-yl}methoxy)-8-azabicyclo[3.2.1]octan-8-yl]pyridine-3-carboxylic acid;
methyl 5-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylate;
methyl 5-[3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylate;
5-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylic acid;
5-[3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylic acid;
2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-6-methylpyrimidine-4-carboxylic acid;
2-[3-({5-cyclopropyl-3-[2-(trifluoromethyl)phenyl]-1,2-oxazol-4-yl}methoxy)-8-azabicyclo[3.2.1]octan-8-yl]-6-methylpyrimidine-4-carboxylic acid;
(2S,3S,4S,5R,6S)-6-((2-((1R,3S,5S)-3-((5-cyclopropyl-3-(2-(trifluoromethyl)phenyl)isoxazol-4-yl)methoxy)-8-azabicyclo[3.2.1]octan-8-yl)-4-fluorobenzo[d]thiazole-6-carbonyl)oxy)-3,4,5-trihydroxytetrahydro-2H-pyran-2-carboxylic acid;
6-((2-(3-((5-cyclopropyl-3-(2-(trifluoromethyl)phenyl)isoxazol-4-yl)methoxy)-8-azabicyclo[3.2.1]octan-8-yl)-4-fluorobenzo[d]thiazole-6-carbonyl)oxy)-3,4,5-trihydroxytetrahydro-2H-pyran-2-carboxylic acid;
(2S,3S,4S,5R,6S)-6-((2-((1R,3S,5S)-3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazole-6-carbonyl)oxy)-3,4,5-trihydroxytetrahydro-2H-pyran-2-carboxylic acid;
6-((2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazole-6-carbonyl)oxy)-3,4,5-trihydroxytetrahydro-2H-pyran-2-carboxylic acid;
(2S,3S,4S,5R,6S)-6-((6-((1R,3S,5S)-3-((5-cyclopropyl-3-(2-(trifluoromethoxy)phenyl)isoxazol-4-yl)methoxy)-8-azabicyclo[3.2.1]octan-8-yl)nicotinoyl)oxy)-3,4,5-trihydroxytetrahydro-2H-pyran-2-carboxylic acid;
6-((6-(3-((5-cyclopropyl-3-(2-(trifluoromethoxy)phenyl)isoxazol-4-yl)methoxy)-8-azabicyclo[3.2.1]octan-8-yl)nicotinoyl)oxy)-3,4,5-trihydroxytetrahydro-2H-pyran-2-carboxylic acid; or

a pharmaceutically acceptable salt thereof.

### Embodiment 11

The use of a compound of Formula (I) or (II) according to Embodiment 1, or the compound of Formula (I) or (II) for use according to Embodiment 1, wherein said compound of Formula (I) or (II) is selected from:
methyl 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylate;
2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid;
methyl 2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylate;
2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylic acid;
2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-methoxy-1,3-benzothiazole-6-carboxylic acid;
methyl 6-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyridine-3-carboxylate;
6-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyridine-3-carboxylic acid;
methyl 5-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylate;
5-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylic acid; and
2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-6-methylpyrimidine-4-carboxylic acid;

or a pharmaceutically acceptable salt thereof.

### Embodiment 12

The use of a compound of Formula (I) or (II) according to any one of Embodiments 1 and 3-11, or the compound of Formula (I) or (II) for use according to any one of Embodiments 2-11, wherein said compound of Formula (I) or (II) is 2-[3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid, or a stereoisomer, enantiomer or pharmaceutically acceptable salt thereof; e.g. 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid, or a pharmaceutically acceptable salt thereof.

### Embodiment 13

The use of a compound of Formula (I) or (II) according to any one of Embodiments 1 and 3-11, or the compound of Formula (I) or (II) for use according to any one of Embodiments 2-11, wherein said compound of Formula (I) or (II) is 2-[3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylic acid, or a stereoisomer, enantiomer or pharmaceutically acceptable salt thereof; e.g., 2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylic acid, or a pharmaceutically acceptable salt thereof.

### Embodiment 14

The use of a compound of Formula (I) or (II) according to any one of Embodiments 1 and 3-11, or the compound of Formula (I) or (II) for use according to any one of Embodiments 2-11, wherein said compound of Formula (I) or (II) is 2-[3-({5-cyclopropyl-3-[2-(trifluoromethoxy)phenyl]-1,2-oxazol-4-yl}methoxy)-8-azabicyclo[3.2.1]octan-8-yl]-4-methoxy-1,3-benzothiazole-6-carboxylic acid, or a stereoisomer, enantiomer or pharmaceutically acceptable salt thereof; e.g., 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-methoxy-1,3-benzothiazole-6-carboxylic acid, or a pharmaceutically acceptable salt thereof.

### Embodiment 15

The use of a compound of Formula (I) or (II) according to any one of Embodiments 1 and 3-11, or the compound of Formula (I) or (II) for use according to any one of Embodiments 2-11, wherein said compound of Formula (I) or (II) is 6-[3-({5-cyclopropyl-3-[2-(trifluoromethoxy)phenyl]-1,2-oxazol-4-yl}methoxy)-8-azabicyclo[3.2.1]octan-8-yl]pyridine-3-carboxylic acid, or a stereoisomer, enantiomer or pharmaceutically acceptable salt thereof; e.g., 6-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyridine-3-carboxylic acid, or a pharmaceutically acceptable salt thereof.

### Embodiment 16

The use of a compound of Formula (I) or (II) according to any one of Embodiments 1 and 3-11, or the compound of Formula (I) or (II) for use according to any one of Embodiments 2-11, wherein said compound of Formula (I) or (II) is 5-[3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylic acid, or a stereoisomer, enantiomer or pharmaceutically acceptable salt thereof; e.g., 5-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylic acid, or a pharmaceutically acceptable salt thereof.

### Embodiment 17

The use of a compound of Formula (I) or (II) according to any one of Embodiments 1 and 3-11, or the compound of Formula (I) or (II) for use according to any one of Embodiments 2-11, wherein said compound of Formula (I) or (II) is 2-[3-({5-cyclopropyl-3-[2-(trifluoromethyl)phenyl]-1,2-oxazol-4-yl}methoxy)-8-azabicyclo[3.2.1]octan-8-yl]-6-methylpyrimidine-4-carboxylic acid, or a stereoisomer, enantiomer or pharmaceutically acceptable salt thereof; e.g., 2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-6-methylpyrimidine-4-carboxylic acid, or a pharmaceutically acceptable salt thereof.

### Embodiment 18

The use of a compound of Formula (I) or (II) according to any one of Embodiments 1 and 3-17, or the compound of Formula (I) or (II) for use in any one of Embodiments 2-17, wherein the condition mediated by FXR is bile acid malabsorption.

### Embodiment 19

The use of a compound of Formula (I) or (II) according to any one of Embodiments 1 and 3-18, or the compound of Formula (I) or (II) for use in any one of Embodiments 2-18, wherein the condition mediated by FXR is primary bile acid diarrhea.

### Embodiment 20

The use of a compound of Formula (I) or (II) according to any one of Embodiments 1 and 3-18, or the compound of Formula (I) or (II) for use in any one of Embodiments 2-18, wherein the condition mediated by FXR is secondary bile acid diarrhea.

### Embodiment 21

The use of a compound of Formula (I) or (II) according to any one of Embodiments 1 and 3-18, or the compound of Formula (I) or (II) for use in any one of Embodiments 2-18, wherein said compound has an EC₅₀ value between 0.1 nM and 500 nM.

### Embodiment 22

The use of a compound of Formula (I) or (II) according to any one of Embodiment 21, wherein said compound has an EC₅₀ value between 0.1 nM and 100 nM.

### Embodiment 23

The use of a compound of Formula (I) or (II) according to any one of Embodiment 21, wherein said compound has an EC₅₀ value between 0.1 nM and 50 nM.

### Embodiment 25

The use of a compound of Formula (I) or (II) according to any one of Embodiment 21, wherein said compound has an EC₅₀ value between 0.1 nM and 30 nM.

### Embodiment 26

The use of a compound of Formula (I) or (II) according to any one of Embodiments 1 and 3-17, in the manufacture of a medicament for treating a condition mediated by Farnesoid X receptor (FXR), wherein said condition is bile acid malabsorption or bile acid diarrhea (e.g. is primary or secondary bile acid diarrhea), bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, or parenteral nutrition-associated liver disease.

### Embodiment 27

A compound of Formula (I) or (II) according to any one of Embodiments 2-17, or a stereoisomer, enantiomer, or a pharmaceutically acceptable salt thereof; and optionally in combination with a second therapeutic agent, for use in treating a condition mediated by FXR; wherein said condition mediated by FXR is bile acid malabsorption or bile acid diarrhea (e.g. primary or secondary bile acid diarrhea), bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, or parenteral nutrition-associated liver disease.

### Embodiment 28

A method for treating or preventing a condition mediated by Farnesoid X receptor (FXR) in a subject suffering therefrom, comprising administering to the subject a therapeutically effective amount of a compound of Formula (I) or (II) or a stereoisomer, enantiomer or pharmaceutically acceptable salt thereof, as described in any of Embodiments 1 and 3-17; and optionally in combination with a second therapeutic agent; wherein said condition mediated by FXR is bile acid malabsorption or bile acid diarrhea (e.g. is primary or secondary bile acid diarrhea), bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, or parenteral nutrition-associated liver disease.

### Embodiment 29

A method for treating or preventing diarrhea or diarrheal disease in a subject suffering therefrom, comprising administering to the subject a therapeutically effective amount of a compound of Formula (I) or (II), or a stereoisomer, enantiomer or pharmaceutically acceptable salt thereof, as described in any of Embodiments 1 and 3-17.

### DETAILED DESCRIPTION OF THE INVENTION

### Definitions

For purposes of interpreting this specification, the following definitions will apply and whenever appropriate, terms used in the singular will also include the plural and vice versa.

As used herein, the term “FXR agonist” refers to an agent that directly binds to and upregulates the activity of FXR.

As used herein, a “stereoisomer” refers to a compound made up of the same atoms bonded by the same bonds but having different three-dimensional structures, which are not interchangeable; and encompasses various stereoisomers (including diastereoisomers and enantiomers), a mixture of stereoisomers or a single stereoisomer.

As used herein, the term “therapeutically effective amount” refers to an amount of the compound of Formula (I) or (II), which is sufficient to achieve the stated effect. Accordingly, a therapeutically effective amount of a compound of Formula (I) or (II) used for the treatment or prevention of a condition mediated by FXR will be an amount sufficient for the treatment or prevention of the condition mediated by FXR.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “diarrhea” or “diarrheal disease” encompasses one, a plurality, or all of the diarrheal subtypes, including those selected from the group consisting of diarrhea associated with inflammatory diseases (e.g., ulcerative colitis, Crohn's disease), infectious diarrheas (e.g., E. Coli, Salmonella, Clostridium difficile, cholera, Campylobacter, rotoviruses etc.), Irritable Bowel Syndrome (specifically, the IBS-D subtype), drug-induced diarrheas (e.g., chemotherapy-induced diarrhea, bile acid-induced diarrhea (e.g., short bowel syndrome, cholecystectomy etc.), diabetic diarrhea (such as those resulting from enteropathy or drug use), allergic diarrhea, diarrhea associated with Celiac disease, and diarrhea associated with Carcinoid syndrome.

### DESCRIPTION OF THE FIGURES

FIG. 1A shows the effect of a compound of Formula (I) (“Compound A”) on serum marker (AST) of cholestasis and liver damage in the chronic treatment rat ANIT model.

FIG. 1B shows the effect of a compound of Formula (I) (“Compound A”) on serum marker (ALT) of cholestasis and liver damage in the chronic treatment rat ANIT model.

FIG. 1C shows the effect of a compound of Formula (I) (“Compound A”) on serum marker (TBIL) of cholestasis and liver damage in the chronic treatment rat ANIT model.

FIG. 1D shows the effect of a compound of Formula (I) (“Compound A”) on serum marker (T) of cholestasis and liver damage in the chronic treatment rat ANIT model.

FIG. 1E shows serum FGF15 protein levels following treatment with a compound of Formula (I) (“Compound A”) in the chronic rat ANIT-induced cholestasis model.

### MODES OF CARRYING OUT THE INVENTION

The present invention provides the use of FXR agonists or partial agonists for treating or preventing liver disease and gastrointestinal disease.

In one aspect, the invention provides the use of a compound of Formula (I)

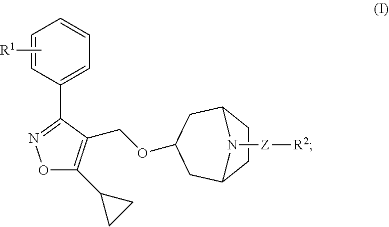

or a stereoisomer, enantiomer, or pharmaceutically acceptable salt thereof;

wherein Z is pyridyl, pyrimidinyl, pyrazinyl, pyridazinyl or benzothiazolyl; each of which is optionally substituted with 1-2 R³ radicals selected from halogen, C₁₋₆ alkyl or C₁₋₆ alkoxy;

R¹ is haloC₁₋₆ alkyl or haloC₁₋₆ alkoxy;

R² is —CO₂R, —CONR—(CR₂)—CO₂R, —CONR—(CR₂)₂—SO₃R or

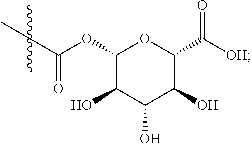 [SMILES: *C(=O)O[C@@H]1O[C@H](C(=O)O)[C@@H](O)[C@H](O)[C@H]1O]

and each R is independently hydrogen or C₁₋₆ alkyl;

or a compound of Formula (II)

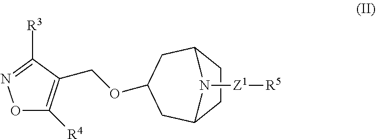

or a stereoisomer, enantiomer, a pharmaceutically acceptable salt, an amino acid conjugate or an acyl glucuronide conjugate thereof;

wherein Z¹ is phenylene, pyridylene, pyrimidinylene, pyrazinylene, pyridazinylene, thiazolylene, benzothiazolyl, benzo[d]isothiazolyl, imidazo[1,2-a]pyridinyl, quinolinyl, 1H-indolyl, pyrrolo[1,2-b]pyridazinyl, benzofuranyl, benzo[b]thiophenyl, 1H-indazolyl, benzo[d]isoxazolyl, quinazolinyl, 1H-pyrrolo[3,2-c]pyridinyl, pyrazolo[1,5-a]pyrimidinyl, imidazo[1,2-b]pyridazinyl, pyrazolo[1,5-a]pyridinyl; each of which is optionally substituted with 1-2 R⁶ radicals selected from halogen, C₁₋₆ alkyl, haloC₁₋₆alkyl, C₁₋₆ alkoxy, haloC₁₋₆alkoxy, or cyclopropyl;

R³ is phenyl, pyridyl, bicyclo[3.1.0]hexanyl, spiro[2.3]hexanyl, bicyclo[3.1.1]heptanyl, spiro[2.5]octanyl, bicyclo[4.1.0]heptanyl, bicyclo[3.1.0]hexan-6-yl, spiro[2.3]hexan-5-yl, bicyclo[3.1.1]heptan-3-yl, spiro[2.5]octan-4-yl, bicyclo[4.1.0]heptan-3-yl, cyclohexyl or cyclopentyl, each of which is optionally substituted with 1-3 R^{3a}; or R³ is cyclopropyl optionally substituted with 1-2 R^{3a} or phenyl;

R^{3a} is halogen, C₁₋₆ alkyl, haloC₁₋₆alkyl, C₁₋₆ alkoxy, haloC₁₋₆alkoxy or cyclopropyl;

R⁴ is C₁₋₃ alkyl, haloC₁₋₃alkyl or cyclopropyl optionally substituted with C₁₋₃ alkyl or haloC₁₋₃ alkyl;

R⁵ is —X—CO₂R⁷, hydroxyC₁₋₆alkyl, CONR⁷R⁸, CONR(CR₂)₁₋₄CO₂R⁷, CONR(CR₂)₁₋₄SO₃R⁸ or tetrazolyl; wherein X is a bond, C₁₋₂ alkylene or cyclopropyl; and

R, R⁷ and R⁸ are independently hydrogen or C₁₋₆ alkyl;

for treating or preventing liver disease or gastrointestinal disease.

In another aspect, the invention provides the use of a compound of Formula (I) or (II), or a stereoisomer, enantiomer or pharmaceutically acceptable salt thereof, in the manufacture of a medicament for treating or preventing liver disease or gastrointestinal disease.

In one embodiment, the invention provides a compound of Formula (I) or (II), or a stereoisomer, enantiomer or pharmaceutically acceptable salt thereof, for use in the treatment or prevention of cholestatic liver disorders, particularly Alagille syndrome, biliary atresia, ductopenic liver transplant rejection, bone marrow or stem cell transplant associated graft versus host disease, cystic fibrosis liver disease and parenteral nutrition-associated liver disease ((PNALD, also known as intestinal failure-associated liver disease).

Parenteral nutrition-associated liver disease (PNALD) is a serious complication of parenteral nutrition (PN) in infants who do not tolerate enteral feedings, especially those with acquired or congenital intestinal diseases. Recent reports have shown that infusion with lipid emulsions derived from fish oil (FO) rather than soy oil (SO) improves established PNALD, and that reduction of the SO lipid dose in PN solutions attenuates PNALD. One of the components of SO emulsions, phylosterol, has been implicated in PNALD. Mechanistic studies have demonstrated that among the phylosterols present in SO emulsions, stigmasterol was by far the most potent at inhibiting activity of FXR, which regulates transcription of bile acid transporters in cultured hepatocytes. On the basis of in vitro studies, stigmasterol has been suggested as promoting cholestasis through inhibition of the nuclear receptor FXR, which, in turn, would result in reduced hepatocyte expression of a wide variety of FXR-dependent genes, including the principal determinant of bile secretion, the bile salt export pump (BSEP) (Abcb11). (Carter et al., Pediatr. Res. 62: 301-306 (2007); El Kasmi et al., Sci. Transl. Med. 5: 1-10 (2013)).

In another embodiment, the invention provides a compound of Formula (I) or (II), or a stereoisomer, enantiomer or pharmaceutically acceptable salt thereof, for use in the treatment or prevention of gastrointestinal diseases, particularly bile acid malabsorption or bile acid diarrhea (including primary bile acid diarrhea and secondary bile acid diarrhea), bile reflux gastritis and inflammatory bowel diseases (IBD), particularly collagenous colitis, lymphocytic colitis, diversion colitis, and indeterminate colitis.

Primary bile acid diarrhea (pBAD) is a common cause of chronic diarrhea, and is characterized by a cycle wherein the feedback regulation of bile acid synthesis is interrupted, resulting in additional bile acid production. Feedback regulation of bile acid synthesis is under the control of an endocrine pathway, wherein activation of the nuclear bile acid receptor FXR induces enteric expression of fibroblast growth factor 15 (FGF15) in rodents and FGF19 in humans. In liver, FGF15 or FGF19 acts together with FXR-mediated expression of small heterodimer partner to repress bile acid synthesis (Jung et al., Journal of Lipid Research 48: 2693-2700 (2007) Walters J R, Nat Rev Gastroenterol Hepatol. 11(7):426-34 (2014)).

Many patients suffering from pBAD have reduced levels of the ileal hormone fibroblast growth factor 19 (FGF19), an inhibitory regulator of hepatic bile acid synthesis, secreted in response to FXR activation. FGF19 production in the ileum is stimulated by bile acid binding to FXR, and activating transcription. Recent studies show that therapy with an FXR agonist significantly increased FGF19 in the primary and secondary BAD group, which were in turn associated with reduced bile acid synthesis and clinical improvement. (Walters J R et al., Nat Rev Gastroenterol Hepatol. 11(7):426-34 (2014); Walters J R et al., Aliment Pharmacol Ther. 2014 Oct. 20. doi: 10.1111/apt. 12999).

Bile acids from duodenogastric reflux promote inflammation and increase the risk for gastro-esophageal cancers. FXR is a transcription factor regulated by bile acids such as CDCA (chenodeoxycholic acid), and protects the liver and the intestinal tract against bile acid overload. (Lian et al., Biochem J. 438: 315-323 (2011)).

Collagenous colitis (CC) is an inflammatory bowel disease (IBD) of unknown origin. In a considerable proportion (44%) of patients with collagenous colitis, the patient suffers from the simultaneous occurrence of bile acid malabsorption. (Ung et al., Gut 46: 170-175 (2000)). Bile acid malabsorption is more uncommon in lymphocytic colitis than in collagenous colitis; however, the 75SeHCAT values suggest a role of bile acids in lymphocytic colitis. The conversion of two patients with lymphocytic colitis to collagenous colitis, and disturbed absorption of bile acids in lymphocytic colitis, suggest that lymphocytic colitis and collagenous colitis represent variants of the same disease. (Ung et al., Hepato-Gastroenterology 49: 432-437 (2002)). FXR activation has also been demonstrated to prevent chemically induced intestinal inflammation, with improvement of colitis symptoms. (Gadaleta et al., Gut 60:463-472 (2011)).

In another aspect, the invention provides the use of a compound of Formula (I) or (II), or a stereoisomer, enantiomer or pharmaceutically acceptable salt thereof, for treating a condition mediated by Farnesoid X receptor (FXR), wherein said condition is bile acid malabsorption (e.g. is primary or secondary bile acid diarrhea), bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, or parenteral nutrition-associated liver disease.

In one embodiment, the compound of Formula (I) or (II) for use in any of the above embodiments has an activity EC₅₀ value between 0.1 nM and 500 nM, which can be determined using assays known in the art such as for example, the GST-FXR LBD co-activator interaction assay described in PCT/US2011/062724. In another embodiment, the compound of Formula (I) or (II) for use in any of the above embodiments has an EC₅₀ value between 0.1 nM and 100 nM; between 0.1 nM and 50 nM; or between 0.1 nM and 30 nM. In yet another embodiment, the compound of Formula (I) or (II) for use in any of the above embodiments has an EC₅₀ value that is <0.1 nM or >500 nM.

The compounds for use in the methods of the invention may be administered either simultaneously with, or before or after, one or more other therapeutic agent. The compound for use in the methods of the invention may be administered separately, by the same or different route of administration, or together in the same pharmaceutical composition as the other agents.

In combination therapies for use in the methods of the invention, a compound of Formula (I) or (II) and the other therapeutic agent may be manufactured and/or formulated by the same or different manufacturers. Moreover, the compound of Formula (I) or (II) and the other therapeutic may be brought together into a combination therapy: (i) prior to release of the combination product to physicians (e.g. in the case of a kit comprising a compound of Formula (I) or (II) and the other therapeutic agent); (ii) by the physician themselves (or under the guidance of the physician) shortly before administration; (iii) in the patient themselves, e.g. during sequential administration of a compound of Formula (I) or (II) and the other therapeutic agent.

Accordingly, the invention provides for the use of a compound of Formula (I) or (II) for treating or preventing a disease or condition mediated by FXR, wherein the medicament is prepared for administration, or administered with, another therapeutic agent. The invention also provides a compound of Formula (I) or (II) for use in a method of treating or preventing a disease or condition mediated by FXR, wherein the compound of Formula (I) or (II) is prepared for administration, or administered with, another therapeutic agent. The invention also provides another therapeutic agent for use in a method of treating or preventing a disease or condition mediated by FXR, wherein the other therapeutic agent is prepared for administration, or administered with, a compound of Formula (I) or (II).

The invention also provides for the use of a compound of Formula (I) or (II) for treating or preventing a disease or condition mediated by FXR, wherein the patient has previously (e.g. within 24 hrs) been treated with another therapeutic agent. Alternatively, the invention provides for the use of another therapeutic agent for treating or preventing a disease or condition mediated by FXR, wherein the patient has previously (e.g. within 24 hrs) been treated with a compound of Formula (I) or (II).

The invention further provides pharmaceutical compositions or combinations comprising a compound of Formula (I) or (II) for treating or preventing liver disease and gastrointestinal disease as described herein. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

In one embodiment, a compound of Formula (I) or (II) is administered at the daily dosage.

In another embodiment, a compound of Formula (I) or (II) is administered enterally; and more particularly, orally.

Unless specified otherwise, a compound for use in the methods of the invention refers to a compound of Formula (I) or (II), pharmaceutically acceptable salt thereof, prodrugs, and inherently formed moieties (e.g., polymorphs, solvates and/or hydrates). The compound for use in the methods of the invention may be stereoisomers (including diastereoisomers and enantiomers), a mixture of stereoisomers or a single stereoisomer, tautomers or isotopically labeled compounds (including deuterium substitutions). Any formula given herein is also intended to represent unlabeled forms as well as isotopically labeled forms of the compounds.

### EXAMPLES

Examples of a compound of Formula (I) or (II) for use in the methods of the present invention are described in PCT/US2011/062724. The following examples are offered to illustrate, but not to limit, the compounds for use in the methods of the present invention.

### Abbreviations

AcOH acetic acid
ANIT alpha-naphthyl-isothiocyanate
ALP alkaline phosphatase
ALT alanine aminotransferase
AST aspartate aminotransferase
EtOAc ethyl acetate
EtOH ethanol
FGF15/19 Fibroblast Growth Factor (known as FGF19 in humans)
GGT gamma-glutamyl transpeptidase
LLQ lower limit of quantification
MeOH methanol
THF tetrahydrofuran
TBA Total bile acids
TBIL Total bilirubin

### Example 1

### 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid (1-1B) and 2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylic acid (1-2B)

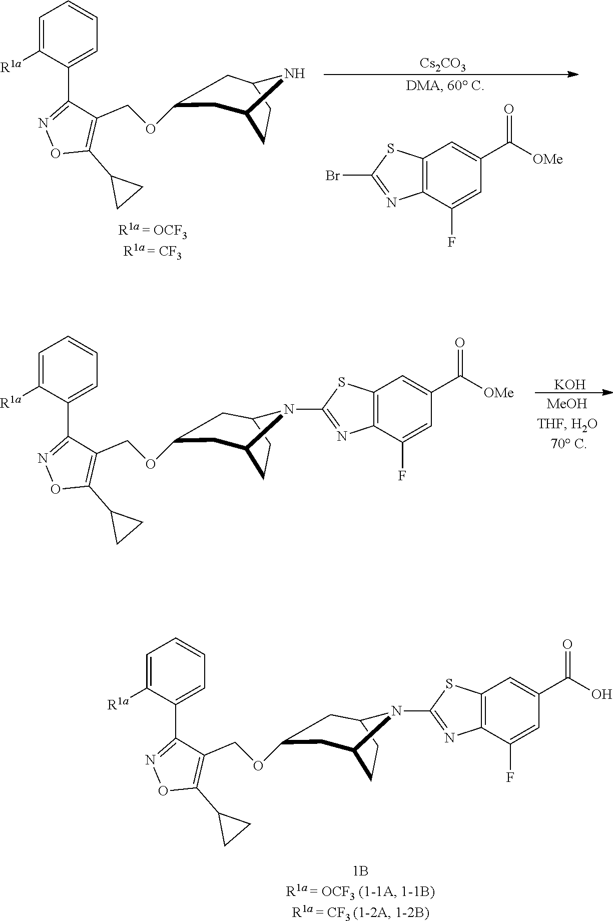

### Methyl 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylate (1-1A)

Into a 25-mL round-bottom flask equipped with a stir bar was added sequentially 4-(((1R,3r,5S)-8-azabicyclo[3.2.1]octan-3-yloxy)methyl)-5-cyclopropyl-3-(2-(trifluoromethoxy)phenyl)isoxazole (1.29 mmol), N,N-dimethylacetamide (3.6 mL), cesium carbonate (3.31 mmol), and methyl 2-bromo-4-fluorobenzo[d]thiazole-6-carboxylate (3.87 mmol). After stirring the resulting slurry at room temperature for 10 minutes, the mixture was then warmed to 60° C. and stirred for 1 h. The reaction slurry was allowed to cool to room temperature, and was diluted with 200 mL of ethyl acetate and washed with water (3×30 mL). The organic extracts were concentrated under vacuum and directly purified using normal phase silica gel chromatography (40 g silica column) with a 15 min gradient of 10% to 60% ethyl acetate/hexanes. Desired fractions were concentrated in vacuo, and the resulting residue crystallized upon standing to give methyl 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylate (I-1A) as a white crystalline solid. MS (m/z): 618.2 (M+1).

### 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid (1-1B)

To a 25-mL round-bottom flask equipped with a stir bar was added the ester (0.89 mmol), THF (4 mL), MeOH (2 mL), and 3 N aqueous KOH solution (1 mL, 3 mmol). The resulting homogenous solution was stirred for 1 hour at 70° C., cooled to room temperature, and then quenched with AcOH (roughly 0.2 mL of glacial acetic, 3 mmol) until pH=6 was achieved (Whatman class pH strip paper). At this time the reaction was diluted with ethyl acetate (40 mL) and washed with water (3×5 mL). The ethyl acetate fraction was concentrated under vacuum to give to an oily residue. To the resulting oil was then added MeOH (6 mL). The oil quickly dissolved, then immediately began to crystallize. Upon standing for 2.5 hrs, the mother liquor was withdrawn and crystals washed (3×2 mL of ice cold MeOH). The crystals were dried via vacuum (10 mm Hg pressure at 45° C. overnight) and then recrystallized from acetonitrile, filtered, and dried under vacuum to give 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid (1-1B). 2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylic acid (1-2B).

Examples 1-2A and the corresponding acid 1-2B can be prepared following the same procedures, from the reaction of intermediate 4-((8-azabicyclo[3.2.1]octan-3-yloxy)methyl)-5-cyclopropyl-3-(2-(trifluoromethyl)phenyl)isoxazole.

| | | Physical Data |
|---|---|---|
| Ex | | MS (m/z), ¹H NMR |
| 1-1A | 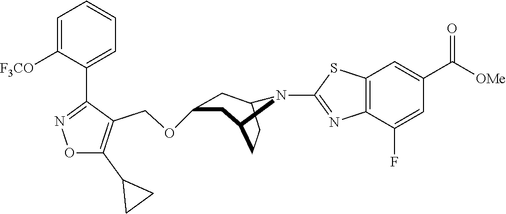 | ¹H NMR (DMSO-d₆, 400 MHz): δ 8.13 (d, J = 1.6 Hz, 1H), 7.67-7.59 (m, 3H), 7.54-7.50 (m, 2H), 4.41 (s, 2H), 4.31 (bs, 2H), 3.90 (s, 3H), 3.60 (t, J = 4.8 Hz, 1H), 2.31-2.25 (m, 1H), 2.10 (app dt, J = 14.8, 4 Hz, 2H), 2.02-1.91 (m, 4H), 1.83 (app d, J = 14.8 Hz, 2H), 1.19-1.15 (m, 4H). MS (m/z): 618.2 (M + 1). |
| 1-1B | 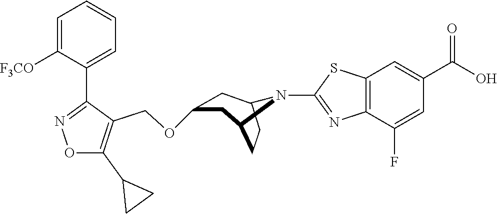 | ¹H NMR (MeOD, 400 MHz): δ 8.03 (d, J = 1.6 Hz, 1H), 7.57-7.53 (m, 2H), 7.49 (dd, J = 8.1, 1.8 Hz, 2H), 7.41 (app t, J = 7.6, 1H), 4.31 (s, 2H), 4.22 (broad s, 2H), 3.50 (t, J = 4.4 Hz, 1H), 2.22-2.15 (m, 1H), 2.00 (app dt, J = 14.8, 4.0 Hz, 2H), 1.91-1.81 (m, 4H), 1.75 (d, J = 14.4, 2H), 1.10-1.05 (m, 4H). MS (m/z): 604.2 (M + 1). |
| 1-2A | 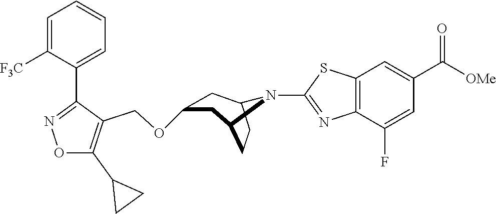 | ¹H NMR (DMSOd₆, 400 MHz): δ 8.26 (d, J = 1.6 Hz, 1H), 7.92 (d, J = 8 Hz, 1H), 7.84-7.74 (m, 2H), 7.63-7.60 (m, 2H), 4.26 (bs, 4H), 3.84 (s, 3H), 3.52 (t, J = 4 Hz, 1H), 2.39-2.31 (m, 1H), 2.01-1.94 (m, 2H), 1.85-1.74 (m, 6H), 1.18-1.06 (m, 4H). MS (m/z): 602.3 (M + 1). |
| 1-2B | 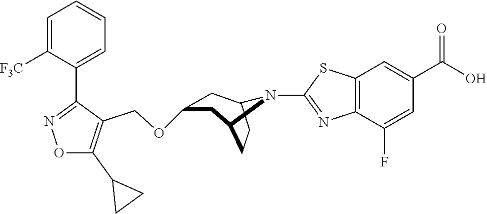 | ¹H NMR (DMSOd₆, 400 MHz): δ 8.21 (d, J = 1.6 Hz, 1H), 7.89 (d, J = 7.2 Hz, 1H), 7.84-7.74 (m, 2H), 7.62-7.56 (m, 2H), 4.26 (bs, 4H), 3.52 (t, J = 4 Hz, 1H), 2.39-2.31 (m, 1H), 2.00-1.96 (m, 2H), 1.85-1.73 (m, 6H), 1.19-1.07 (m, 4H). MS (m/z): 588.1 (M + 1). |

### Example 2

### 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-methoxy-1,3-benzothiazole-6-carboxylic acid

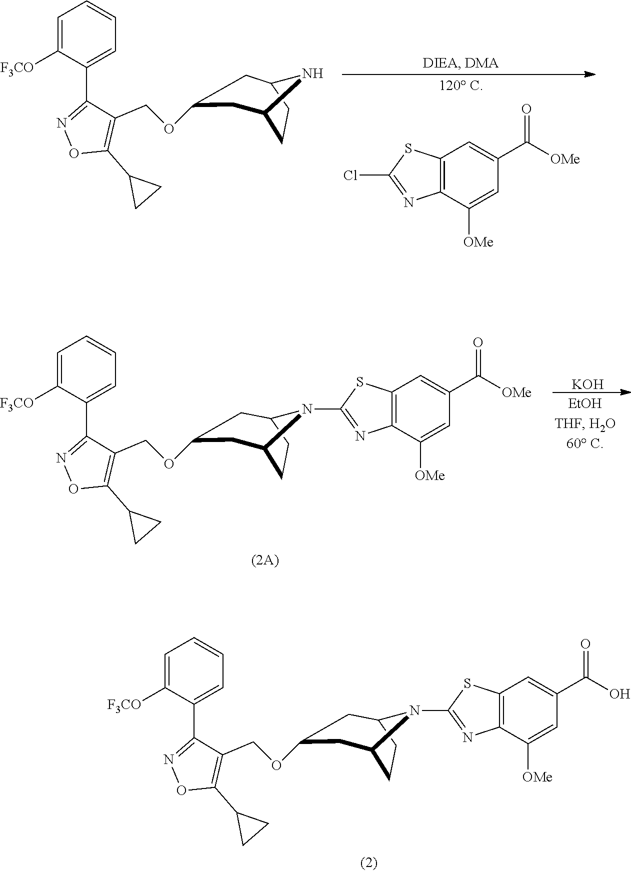

Methyl 2-chloro-4-methoxybenzo[d]thiazole-6-carboxylate (0.48 mmol) and 4-(((1R,3r,5S)-8-azabicyclo[3.2.1]octan-3-yloxy)methyl)-5-cyclopropyl-3-(2-(trifluoromethoxy)phenyl)isoxazole (0.48 mmol) and diisopropylethylamine (0.1 mL, 0.7 mmol) were sequentially dissolved in dimethylacetamide (1 mL) and heated to 120° C. overnight. The reaction mixture was cooled to room temperature and then diluted with ethyl acetate and aqueous saturated sodium bicarbonate solution. The organics were separated, the aqueous layer was subjected to a further wash with ethyl acetate, and the organics were combined and dried (MgSO₄) then evaporated in vacuo. Methyl 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-methoxy-1,3-benzothiazole-6-carboxylate (2A) was obtained as a clear oil after purification by silica gel chromatography with a gradient of 0-100% ethyl acetate/hexanes. MS (m/z): 630.1 (M+1).

The ester (2A) (0.26 mmol) was dissolved in tetrahydrofuran (1 mL) and ethanol (1 mL) and subjected to an aqueous solution of potassium hydroxide (2.5 mmol in 2 mL water). The mixture was heated to 60° C. for 2 hr and then the solvent was removed in vacuo. The mixture was diluted with 5% aqueous citric acid and extracted with ethyl acetate (2×100 mL). The organics were dried (MgSO₄) then evaporated in vacuo. The product was purified by flash silica chromatography with a gradient of 0-100% ethyl acetate/hexanes to give the corresponding acid (2). ¹H NMR (MeOD, 400 MHz): δ 8.77 (s, 2H), 7.66-7.58 (m, 2H), 7.51 (app t, J=8.0 Hz, 2H), 4.63 (bs, 2H), 4.40 (s, 2H), 3.55 (t, J=4.4 Hz, 1H), 2.31-2.24 (m, 1H), 1.99-1.88 (m, 4H), 1.86-1.81 (m, 2H), 1.76 (d, J=14.0 Hz, 2H), 1.19-1.15 (m, 4H). MS (m/z): 616.1 (M+1).

### Example 3

The following compounds were prepared from 4-((8-azabicyclo[3.2.1]octan-3-yloxy)methyl)-5-cyclopropyl-3-(2-(trifluoromethoxy)phenyl)isoxazole and commercially available ethyl 2-chlorobenzo[d]thiazole-6-carboxylate according to the procedures described for the preparation of Example 1 or 2.

| | | Physical Data |
|---|---|---|
| Ex | | MS (m/z), ¹H NMR |
| 3A | 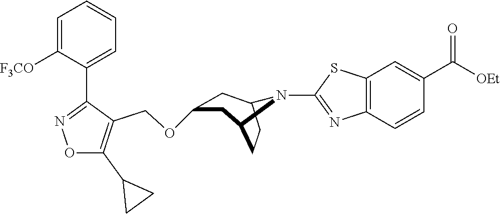 | ¹H NMR (DMSO-d₆, 400 MHZ): δ 8.37 (d, J = 1.6 Hz, 1H), 7.85 (dd, J = 8.8, 2 Hz, 1H), 7.71-7.63 (m, 2H), 7.59-7.53 (m, 2H), 7.47 (d, J = 8.4 Hz, 1H), 4.34 (s, 2H), 4.29 (app q J = 7.2 Hz, 2H), 4.22 (s, 2H), 3.56 (t, J = 4.4 Hz, 2H), 2.39-2.32 (m, 1H), 1.98 (dt, J = 14.8, 4 Hz, 2H), 1.85-1.80 (m, 4H), 1.74 (d, J = 14.4 Hz, 2H), 1.32 (t, J = 7.2 Hz, 3H), 1.17-1.06 (m, 4H). MS (m/z): 614.2 (M + 1). |
| 3B | 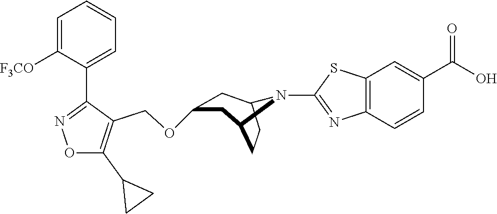 | ¹H NMR (DMSO-d₆, 400 MHz): δ 8.30 (d, J = 1.6 Hz, 1H), 7.81 (dd, J = 8.4, 1.8 Hz, 1H), 7.71-7.62 (m, 2H), 7.60-7.53 (m, 2H), 7.42 (d, J = 8.4 Hz, 1H), 4.33 (s, 2H), 4.19 (bs, 2H), 3.54 (t, J = 4.4 Hz, 1H), 2.39-2.31 (m, 1H), 1.98 (dt, J = 14.8, 4 Hz, 2H), 1.86- 1.77 (m, 4H), 1.73 (app d, J = 16.4 Hz, 2H), 1.17-1.04 (m, 4H). MS (m/z): 586.2 (M + 1). |

### Example 4

### 2-({2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-1,3-benzothiazol-6-yl}formamido)acetic acid (4-1)

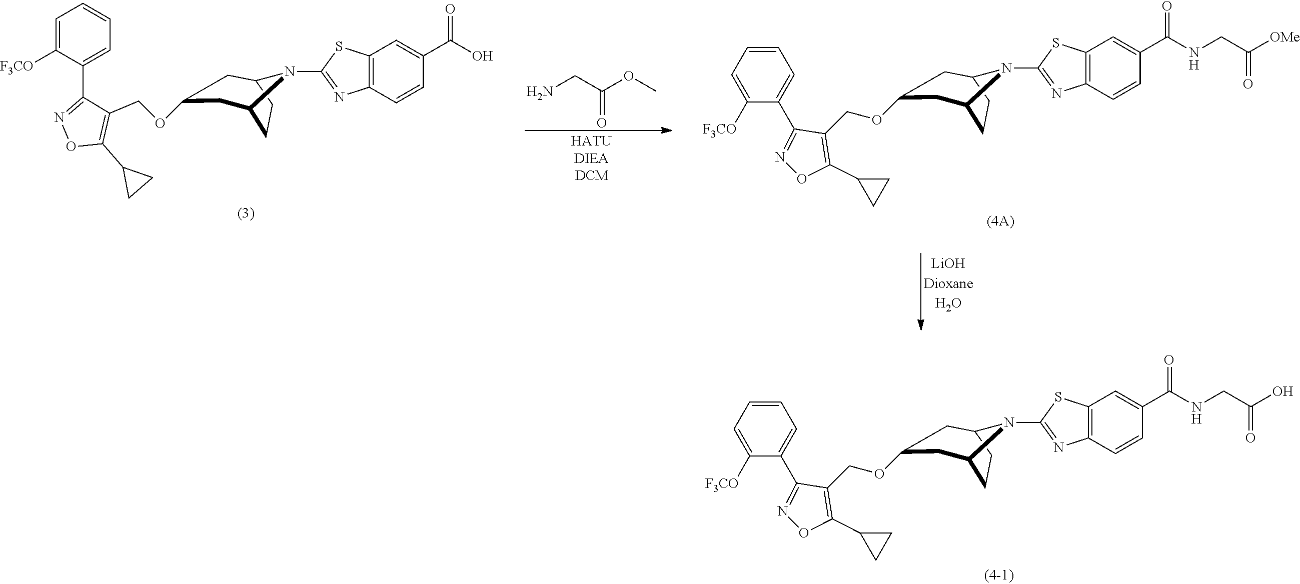

2-((1R,3r,5S)-3-((5-cyclopropyl-3-(2-(trifluoromethoxy)phenyl)isoxazol-4-yl)methoxy)-8-azabicyclo[3.2.1]octan-8-yl)benzo[d]thiazole-6-carboxylic acid (Example 3) (0.06 mmol) was combined with glycine methyl ester hydrochloride (0.06 mmol), HATU (O-(7-Azabenzotriazol-1-yl)-N,N,N′,N′-tetramethyluronium hexafluorophosphate) (0.065 mmol), diisopropylethylacetate (0.05 ml) and dichloromethane (2 mL). The mixture was stirred for 1 hour, then the solvent was removed in vacuo. The residue was suspended in ethyl acetate (15 mL) and washed with sodium bicarbonate solution (5 mL). The organics were combined and dried (MgSO₄) then evaporated in vacuo. The crude product was purified by flash silica chromatography with 0-100% ethyl acetate in hexanes to give the ester (4A).

The ester (4A) was subjected to a solution of 4N LiOH in water (2 mL) and dioxane (2 mL) and stirred for 2 hours. The solvent was reduced in vacuo and the mixture diluted with 5% citric acid (10 mL) and extracted with ethyl acetate (2×8 mL). The organics were combined and dried (MgSO₄) then evaporated in vacuo. The product was purified with flash silica chromatography with methanol/dichloromethane with a 0-40% gradient to give the title compound as a white solid.

Examples 4-2, 4-3 and 4-4 can be prepared following the same procedures, using appropriate intermediates.

| | | Physical Data |
|---|---|---|
| Ex | | MS (m/z), 1H NMR |
| 4-1 | 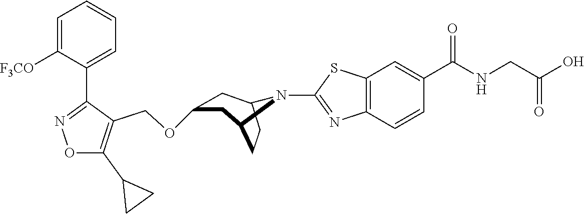 | ¹H NMR (MeOD, 400 MHz): δ 8.07 (d, J = 1.6 Hz, 1H), 7.71 (dd, J = 8.4, 1.6 Hz, 1H), 7.57-7.48 (m, 2H), 7.41 (app t, J = 7.6 Hz, 2H), 7.35 (d, J = 8.4 Hz, 1H), 4.31 (s, 2H), 4.16 (bs, 2H), 3.95 (s, 2H), 3.50 (t, J = 4.4 Hz, 1H), 2.21-2.15 (m, 1H), 2.00 (dt, J = 14.8, 4 Hz, 2H), 1.91-1.81 (m, 4H), 1.72 (d, J = 14.8 Hz, 2H), 1.09-1.05 (m, 4H). MS (m/z): 643.2 (M + 1). |
| 4-2 | 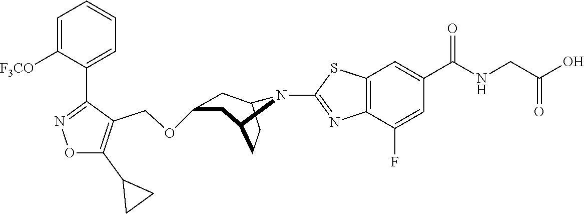 | MS (m/z): 661.2 (M + 1) |
| 4-3 | 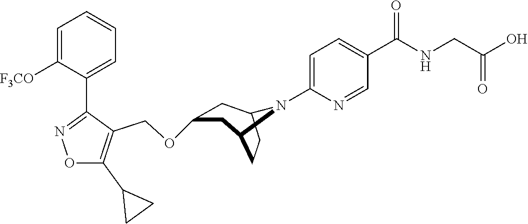 | ¹H NMR (MeOD-d₄, 400 MHz): δ 8.35 (d, J = 2.0 Hz, 1H), 8.29 (dd, J = 9.6, 3.0 Hz, 1H), 7.64 (app dt, J = 7.6, 2.0 Hz, 1H), 7.59 (app dd, J = 8.4, 2.0 Hz, 1H), 7.54-7.48 (m, 2H), 7.28 (d, J = 9.6 Hz, 1H), 4.56 (br s, 2H), 4.44 (s, 2H), 4.18 (s, 2H), 3.61 (app t, J = 4.4 Hz, 1H), 2.29-2.27 (m, 1H), 2.06-1.89 (m, 8H), 1.19-1.15 (m, 4H). MS (m/z): 587.2 (M + 1). |
| 4-4 | 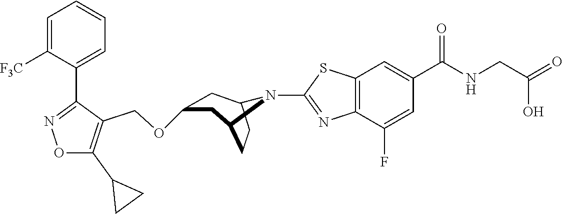 | MS (m/z): 645.1 (M + 1) |

### Example 5

### 2-({2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-1,3-benzothiazol-6-yl}formamido)ethane-1-sulfonic acid (5-1)

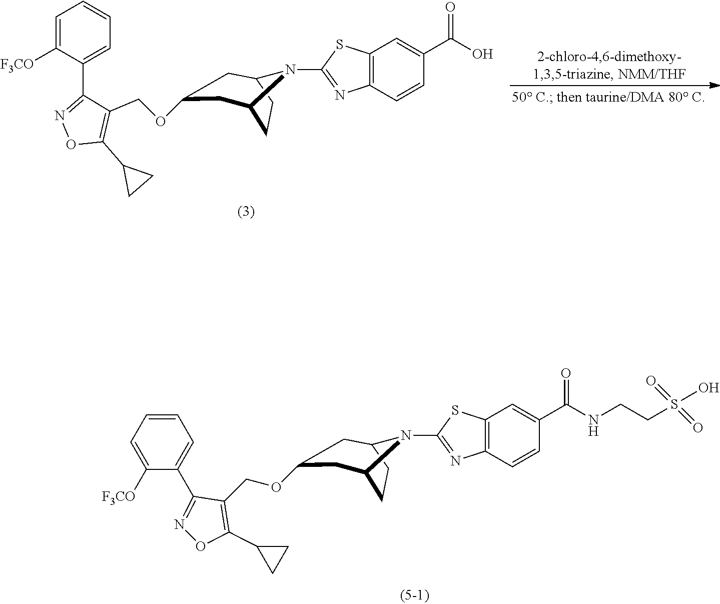

To a resealable and pressure tolerable vessel was added the following in sequential order: 2-((1R,3r,5S)-3-((5-cyclopropyl-3-(2-(trifluoromethoxy)phenyl) isoxazol-4-yl)methoxy)-8-azabicyclo[3.2.1]octan-8-yl)benzo[d]thiazole-6-carboxylic acid (Example 3) (0.1 mmol), tetrahydrofuran (1.0 mL), N-methyl morpholine (approximately 0.1 mL, 0.7 mmol). The suspension was stirred at room temperature for a few minutes until complete dissolution of the starting acid. Next was added 2-chloro-4,6-dimethoxy-1,3,5-triazine (0.15 mmol). and the resulting solution was stirred at 50° C. for 20 minutes until a fine white precipitate formed. This precipitate was physically agitated to ensure that all materials were thoroughly mixed. Next the taurine (0.40 mmol) was added as a dimethyl acetamide (4 mL) suspension. The resulting suspension was sealed in the vessel and heated to 80° C. for 2 hours. The mixture was then cooled to room temperature, diluted with ethyl acetate 20 mL and washed with water (2×3 mL). The organics were dried under vacuum, the resulting residue was diluted with 3 mL of MeOH, and the liquid was directly purified using mass-directed reverse phase HPLC using gradient of 20 to 70% acetonitrile/water with ammonium acetate (0.05%) as modifier. The resulting product was cold vacuum concentrated to give the title compound as a white powder.

Examples 5-2, 5-3 and 5-4 can be prepared following the same procedures, using appropriate intermediates.

| | | Physical Data |
|---|---|---|
| Ex | | MS (m/z), ¹H NMR |
| 5-1 | 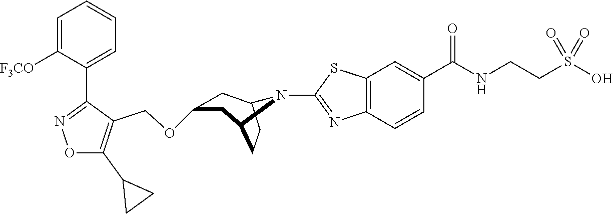 | ¹H NMR (MeOD, 400 MHz): δ 8.27 (d, J = 1.6 Hz, 1H), 8.00 (dd, J = 8.4, 1.9 Hz, 1H), 7.65-7.53 (m, 2H), 7.42 (app t, J = 7.8 Hz, 2H), 7.35 (d, J = 8.4 Hz, 1H), 4.42 (br s, 4H), 3.81 (t, J = 6.4 Hz, 2H), 3.11 (t, J = 6.4 Hz, 2H), 2.18-1.93 (m, 8H), 1.72 (d, J = 14.8 Hz, 2H), 1.29-1.15 (m, 4H). MS (m/z): 693.2 (M + 1). |
| 5-2 | 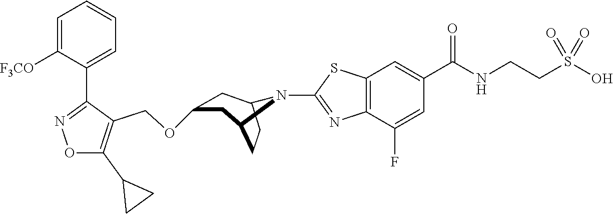 | MS (m/z): 711.2 (M + 1) |
| 5-3 | 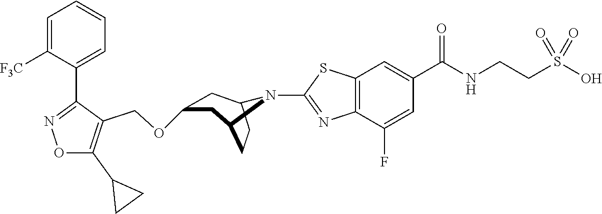 | MS (m/z): 695.3 (M + 1) |
| 5-4 | 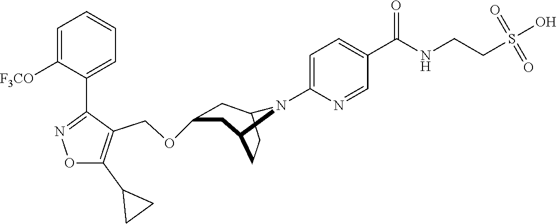 | ¹H NMR (MeOH-d₄, 400 MHz): δ 8.30 (d, J = 2.0 Hz, 1H), 8.26 (d, J = 9.6 Hz, 1H), 7.66 (app dt, J = 7.6, 2.0 Hz, 1H), 7.61 (app dd, J = 8.4, 2.0 Hz, 1H), 7.54-7.49 (m, 2H), 7.25 (dd, J = 9.4, 1.8 Hz, 1H), 4.55 (br s, 2H), 4.49 (s, 2H), 3.78 (t, J = 8.4 Hz, 2H), 3.60 (app t, J = 4.4 Hz, 1H), 3.07 (t, J = 8.4 Hz, 2H), 2.27-2.21 (m, 1H), 2.08-1.93 (m, 8H), 1.21-1.16 (m, 4H). MS (m/z): 637.2 (M + 1). |

### Example 6

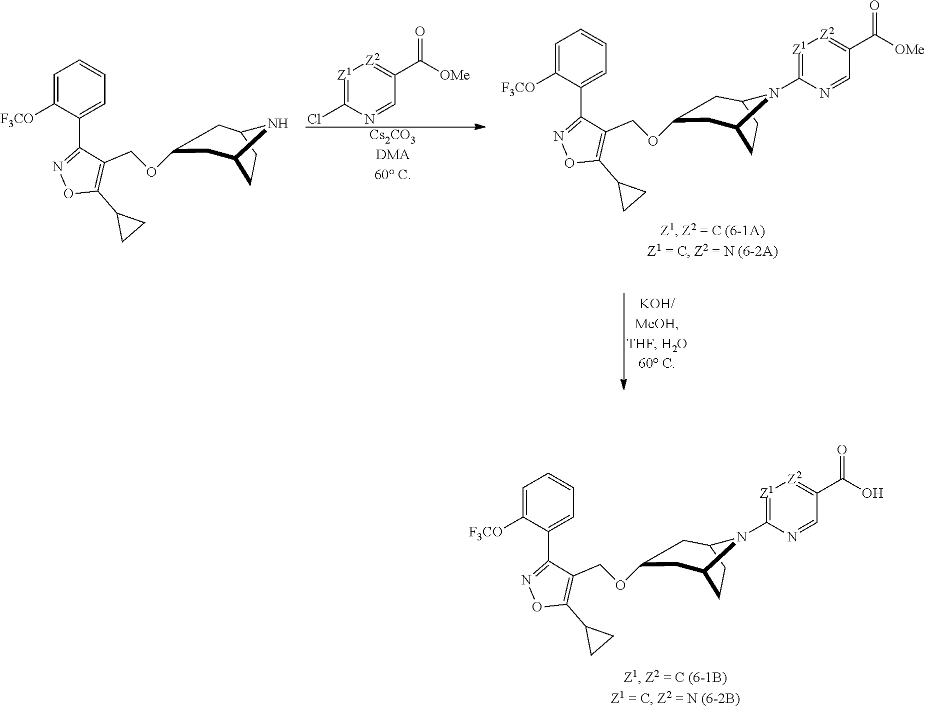

The following examples can be prepared from the reaction of 4-(((1R,3r,5S)-8-azabicyclo[3.2.1]octan-3-yloxy)methyl)-5-cyclopropyl-3-(2-(trifluoromethoxy)phenyl)isoxazole (I-1) and the corresponding pyridyl and pyrazinyl derivative following the procedures described in Example 1.

| | | Physical Data |
|---|---|---|
| Ex | | MS (m/z), ¹H NMR |
| 6-1A | 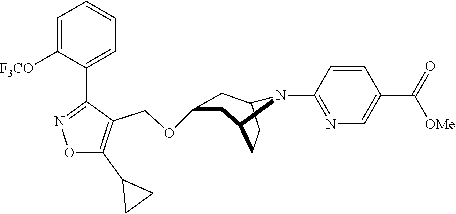 | MS (m/z): 544.2 (M + 1) |
| 6-1B | 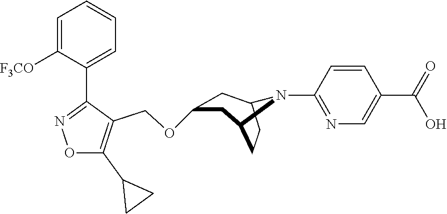 | ¹H NMR (MeOD, 400 MHz): δ 8.64 (d, J = 2 Hz, 1H), 7.98 (dd, J = 8.8, 2.4 Hz, 1H), 7.66-7.58 (m, 2H), 7.52-7.48 (m, 2H), 6.64 (d, J = 9.2 Hz, 1H), 4.43 (bs, 2H), 4.38 (s, 2H), 3.52 (t, J = 4.4 Hz, 1H), 2.31- 2.24 (m, 1H), 1.97-1.82 (m, 6H), 1.71 (d, J = 14.4 Hz, 2H), 1.18-1.14 (m, 4H). MS (m/z): 530.2 (M + 1). |
| 6-2A | 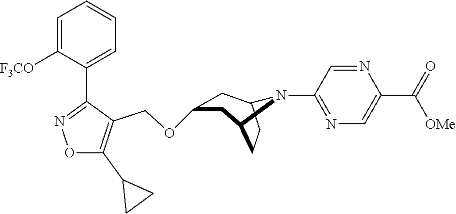 | MS (m/z): 545.2 (M + 1) |
| 6-2B | 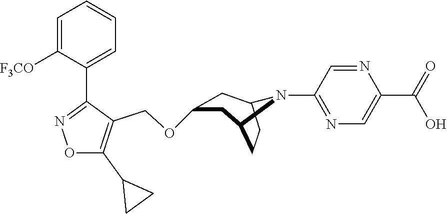 | ¹H NMR (DMSO-d₆, 400 MHz): δ 8.51 (s, 1H), 8.01 (s, 1H), 7.64-7.56 (m, 2H), 7.51-7.46 (m, 2H), 4.41 (bs, 2H), 4.26 (s, 2H), 3.40 (t, J = 4 Hz, 1H), 2.31-2.24 (m, 1H), 1.75-1.67 (m, 6H), 1.57 (d, J = 14.8 Hz, 2H), 1.09-0.97 (m, 4H). MS (m/z): 531.2 (M + 1). |

### Example 7

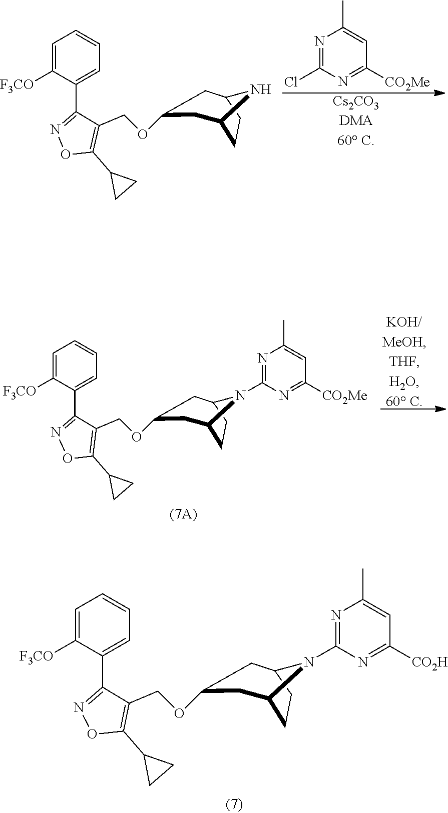

Example 7 was prepared following the procedures in Example 1 from 4-((8-azabicyclo[3.2.1]octan-3-yloxy)methyl)-5-cyclopropyl-3-(2-(trifluoromethyl)phenyl)isoxazole (I-2) and the corresponding pyrimidyl reagent.

| | | Physical Data |
|---|---|---|
| Ex | | MS (m/z), ¹H NMR |
| 7A | 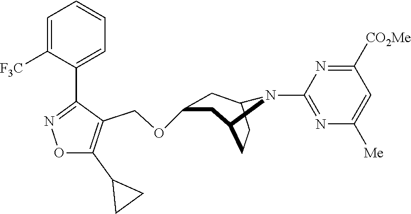 | MS (m/z): 515.3 (M + 1) |
| 7B | 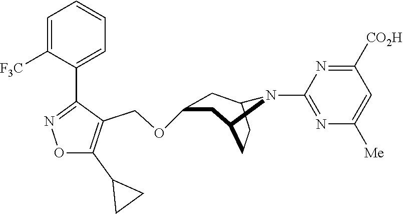 | ¹H NMR (DMSO-d₆, 400 MHz): δ 7.92 (d, J = 7.6 Hz, 1H), 7.83-7.73 (m, 2H), 7.60 (d, J = 7.6 Hz, 1H), 6.94 (s, 1H), 4.50 (bs, 2H), 4.23 (bs, 2H), 3.47-3.40 (m, 1H), 2.38-2.29 (m, 4H), 1.81-1.61 (m, 8H), 1.16-1.04 (m, 4H). MS (m/z): 529.3 (M + 1). |

### Example 8

The following examples were prepared according to the procedures described in Kittelmann, M. et al., Adv. Synth. Catal. 2003, 345, 825-829.

| | | Physical Data |
|---|---|---|
| Ex | | MS (m/z), ¹H NMR |
| 8-1 | 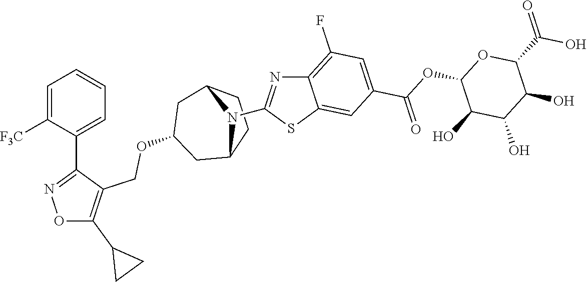 [SMILES: O=C(O[C@@H]1O[C@H](C(=O)O)[C@@H](O)[C@H](O)[C@H]1O)c1cc(F)c2nc(N3[C@@H]4CC[C@H]3C[C@@H](OCc3c(-c5ccccc5C(F)(F)F)noc3C3CC3)C4)sc2c1] | ¹H NMR (DMSO-d₆, 600 MHz): δ 12.87 (br s, 1H), 8.31 (d, J = 1.2 Hz, 1H), 7.91 (app t, J = 8.0 Hz, 1H), 7.76 (app t, J = 8.0 Hz, 1H), 7.71 (d, J = 9.3 Hz, 1H), 7.61 (d, J = 8.0 Hz, 1H), 5.58 (d, J = 7.3 Hz, 1H), 4.25 (s, 4H), 3.83 (d, J = 8.7 Hz, 1H), 3.52 (app t, J = 4.0 Hz, 1H), 3.43-3.31 (m, 4H), 2.36-2.32 (m, 1H), 1.96 (dt, J = 14.0, 4.0 Hz, 2H), 1.85-1.73 (m, 6H), 1.18-1.04 (m, 4H). MS (m/z): 764.3 (M + 1). |
| 8-2 | 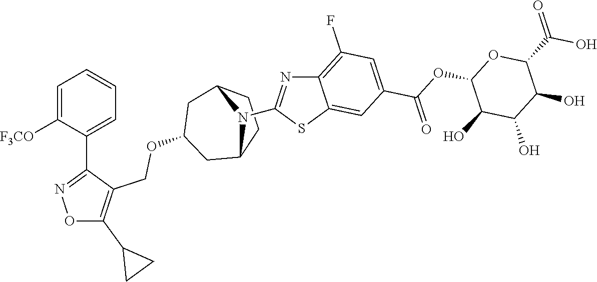 [SMILES: O=C(O[C@@H]1O[C@H](C(=O)O)[C@@H](O)[C@H](O)[C@H]1O)c1cc(F)c2nc(N3[C@@H]4CC[C@H]3C[C@@H](OCc3c(-c5ccccc5OC(F)(F)F)noc3C3CC3)C4)sc2c1] | MS (m/z): 780.2 (M + 1) |
| 8-3 | 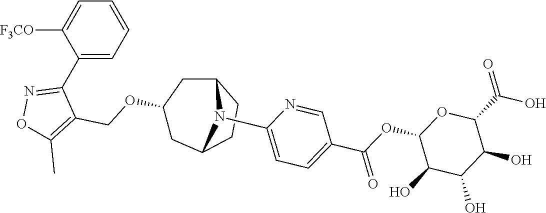 | MS (m/z): 706.3 (M + 1) |

### Example 9

### Effect of Test Compound in Chronic Treatment Rat ANIT Model

A compound of Formula (I) was evaluated in a chronic treatment model of cholestasis over a range of doses from 0.01 to 3 mg/kg. Rats were treated with ANIT (0.1% w/w) in food for 3 days prior to treatment with Compound A at the indicated doses (“Veh”). A non-cholestatic control group was fed standard chow diet without ANIT, and serve as the non-cholestatic control animals (“Control”). After 14 days of oral dosing, the indicated analyte was measured in serum. LLQ, lower limit of quantitation. Mean±SEM; n=5.

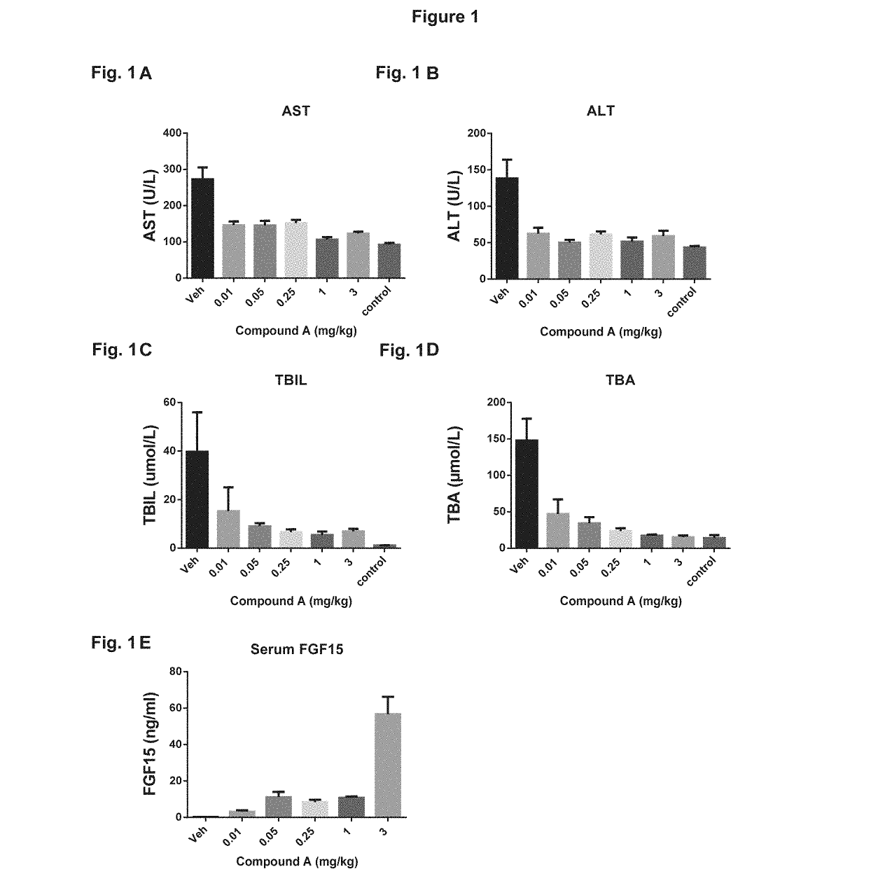

ANIT treatment caused elevation of hepatobiliary injury indicators, such as elevated levels of circulating aspartate aminotransferase (AST) (FIG. 1A), alanine aminotransferase (ALT) (FIG. 1B), bilirubin (FIG. 1C) and bile acids (FIG. 1D) (“Veh” vs “Control”). These data demonstrate that ANIT exposure induced profound cholestasis and hepatocellular damage. In contrast, Compound A improved many of these indicators starting at doses as low as 0.01 mg/kg. Marked reductions of serum bile acid and bilirubin concentrations were observed upon treatment with Compound A. The reduced levels of total bile acids (TBA) levels associated with treatment of Compound A were consistent with the pharmacological action of FXR agonist by reducing accumulation of bile acids in the liver, enhancing bile acid excretion in the biliary tract and inhibiting bile acid synthesis. The improvement in the serum conjugated bilirubin (a direct indicator for hepatic function) by Compound A implies recovery from cholestasis with improved bile excretion.

Furthermore, Compound A stimulated serum FGF15 expression in the chronic treatment rat ANIT model in a dose dependent manner (FIG. 1E). Serum FGF15 levels were quantified using an FGF15 Meso Scale Discovery (MSD) assay. Mouse FGF15 antibody from R&D Systems (AF6755) was used both as capture and detection antibody in the assay. MSD SULFO-TAG NHS-Ester was used to label the FGF15 antibody. MSD standard 96-well plates were coated with the FGF15 capture antibody and the plates were blocked with MSD Blocker A (R93AA-2). After washing the plate with PBS+0.05% Tween 20, MSD diluent 4 was dispensed into each well and incubated for 30 min. 25 μl of calibrator dilutions or samples (serum or EDTA plasma) were dispensed into each well and incubated with shaking at RT. After washing, detection antibody was added and incubated with shaking for 1 h at RT. After washing and the addition of MSD Read buffer (R92TC-2), the plate was read on an MSD SECTOR Imager 6000. Plots of the standard curve and unknown samples were calculated using MSD data analysis software.

Activation of FXR in the ileum induces the expression of fibroblast growth factor 15 (FGF15 in rodent; FGF19 in human), a hormone that is secreted in the portal blood and signals to the liver to repress Cyp7a1 expression synergistically with SHP. The direct FXR-dependent induction of FGF15/19 along with FGF15/19's anti-cholestatic properties makes it a convenient serum biomarker for detecting target engagement of FXR agonists. Significant dose-dependent induction of FGF15 observed with treatment of Compound A demonstrate FXR target engagement by Compound A.

The results demonstrated in FIG. 1 are consistent with the use of a compound of Formula (I) for the treatment of cholestatic liver disorders such as bile acid malabsorption (e.g., primary or secondary bile acid diarrhea), bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, and parenteral nutrition-associated liver disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. The method according to claim 17 wherein R¹ is trifluoromethyl or trifluoromethoxy.

2. The method according to claim 17 wherein R² is —CO₂R, and R is hydrogen or C₁₋₆alkyl.

3. The method according claim 17 wherein R³ is methyl, methoxy or fluoro.

4. The method according to claim 17 wherein Z is pyridyl.

5. The method according to claim 17 wherein Z is pyrimidinyl.

6. The method according to claim 17 wherein Z is pyrazinyl.

7. The method according to claim 17 wherein said Z is benzothiazolyl.

8. The method according to claim 17 wherein said compound of Formula (I) is selected from
methyl 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylate;
2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid;
methyl 2-[(1R,3r, 5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylate;
2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylic acid;
2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-methoxy-1,3-benzothiazole-6-carboxylic acid;
methyl 6-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyridine-3-carboxylate;
6-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyridine-3-carboxylic acid;
methyl 5-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylate;
5-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylic acid; and
2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-6-methylpyrimidine-4-carboxylic acid;
or a pharmaceutically acceptable salt thereof.

9. The method according to claim 17 wherein said compound is 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid, or a pharmaceutically acceptable salt thereof.

10. The method according to claim 17 wherein said compound is 2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-4-fluoro-1,3-benzothiazole-6-carboxylic acid, or a pharmaceutically acceptable salt thereof.

11. The method according to claim 17 wherein said compound is 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-methoxy-1,3-benzothiazole-6-carboxylic acid, or a pharmaceutically acceptable salt thereof.

12. The method according to claim 17 wherein said compound is 6-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyridine-3-carboxylic acid, or a pharmaceutically acceptable salt thereof.

13. The method according to claim 17 wherein said compound is 5-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]pyrazine-2-carboxylic acid, or a pharmaceutically acceptable salt thereof.

14. The method according to claim 17 wherein said compound is 2-[(1R,3r,5S)-3-({5-cyclopropyl-3-[2-(trifluoromethyl)phenyl]-1,2-oxazol-4-yl}methoxy)-8-azabicyclo[3.2.1]octan-8-yl]-6-methylpyrimidine-4-carboxylic acid, or a pharmaceutically acceptable salt thereof.

15. The method according to claim 17 wherein said condition mediated by FXR is bile acid malabsorption.

16. The method according to claim 15, or the compound of Formula (1) for use according to claim 15, wherein said bile acid malabsorption is primary or secondary bile acid diarrhea.

17. A method for treating or preventing a condition mediated by Farnesoid X receptor (FXR) including bile acid malabsorption, bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, and parenteral nutrition-associated liver disease, the method comprising administering to a subject a compound of Formula (I) 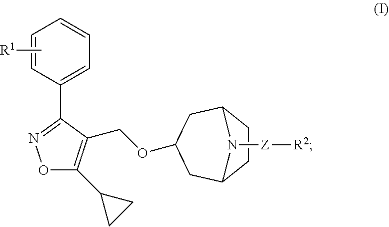
or a stereoisomer, enantiomer, or pharmaceutically acceptable salt thereof;
wherein 7 is pyridyl, pyrimidinyl, pyrazinyl, pyridazinyl or benzothiazolyl; each of which is optionally substituted with 1-2 R³ radicals selected from halogen, C₁₋₆ alkyl or C₁₋₆ alkoxy;
R¹ is haloC₁₋₆alkyl or haloC₁₋₆alkoxy;
R² is —CO₂R, —CONR—(CR₂)—CO₂R, —CONR—(CR₂)₂—SO₃R or 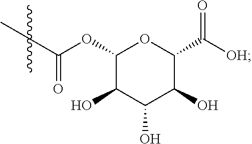 [SMILES: *C(=O)O[C@@H]1O[C@H](C(=O)O)[C@@H](O)[C@H](O)[C@H]1O]
each R is independently hydrogen or C₁₋₆alkyl.

18. The method according to claim 17 further including a second therapeutic agent.
